(19) United States
(12) Patent Application Publication
(10) Pub. No.: US 2005/0131475 A1
(43) Pub. Date: Jun. 16, 2005

(54) Precordial-superior vena cava electrode arrangement for an implantable cardioverter defibrillator

(76) Inventor: Karel F.A.A. Smits, Munstergeleen (NL)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/985,341

(22) Filed: Nov. 9, 2004

Related U.S. Application Data
(63) Continuation of application No. 10/360,762, filed on Dec. 7, 2001, now abandoned.

(51) Int. Cl. A61N 1/39

(52) U.S. Cl. 607/14; 607/5

(57) ABSTRACT

The present invention provides systems, methods, components and medical devices for defibrillating a volume of excitable myocardial tissue. In one aspect, the invention delivers therapy via a pair of electrodes spaced from myocardial tissue with a first electrode disposed in a precordial location and spaced from myocardial tissue and a second electrode disposed in a non-precordial location. Another form of the invention includes methods of delivering cardioversion, defibrillation and/or cardiac pacing therapy by sensing cardiac events with at least a pair of electrodes located in one of a submuscular and a subcutaneous position. In this form of the invention the pair of electrodes are spaced from excitable heart tissue of the subject and cardiac therapy pulses are delivered via at least a pair of electrodes at a sufficient energy level to capture at least one ventricular chamber of the subject.

### RELATED APPLICATION

The present application is a continuation of utility U.S. patent application Ser. No. 10/360,762, filed Dec. 7, 2001, which is a converted non-provisional of provisional U.S. patent application Ser. No. 60/336,728, filed 7 Dec. 2001, the entire disclosures of which are incorporated herein by reference.

### FIELD OF THE INVENTION

The invention relates to an electrode configuration for an implantable cardioverter defibrillator, not requiring, however allowing, a defibrillation electrode placed in a ventricle of the heart.

### BACKGROUND OF THE INVENTION

In the prior art (as depicted in FIG. 1) format a standard arrangement for an implantable cardioverter-defibrillator (ICD) oftentimes includes an optional high voltage (e.g., coil-type) electrode disposed within a portion of the superior vena cava (SVC) part of the vasculature of a patient and/or a high voltage electrode disposed within the right ventricular (RV) chamber. Such high voltage coils disposed can be coupled to a single elongated medical electrical lead and individually controlled via operative circuitry disposed within the ICD can, or housing, thereby providing a selectable high voltage defibrillation or lower voltage cardioversion therapy delivery vector through a volume of myocardial tissue. As is known in the art, the selectable vector is defined as a conductive path through the myocardium from one of the high voltage electrodes to an electrically conductive portion of the can, or housing, for an ICD or between the two high voltage electrodes.

The electrically conductive portion of the can comprise, for example, one or more discrete electrodes such as the apparatus and methods described and depicted in U.S. Pat. No. 6,230,059 to Duffin, assigned to Medtronic, Inc. This patent issued on 8 May 2001 and is entitled, “Implantable Monitor” and sets forth prior art and inventive techniques for collecting physiologic information from a subject via a plurality of subcutaneous and submuscular electrode pairs that define diverse sensing vectors.

In addition, the techniques for discriminating potentially lethal from non-lethal arrhythmias, so as to withhold a high voltage therapy, have been utilized in the art. For example, U.S. Pat. No. 5,545,186 to Olson et al. which is also assigned to Medtronic, Inc. filed on 30 Mar. 1995, issued 13 Aug. 1996 and entitled, “Prioritized Rule Based Method and Apparatus for Diagnosis and Treatment of Arrhythmias” provides one such example known to those of skill in the art to which the invention is directed.

Of course, over the long history of defibrillators used to provide acute therapy (e.g., using temporarily implanted leads and external circuitry) and ambulatory implantable medical devices, such as ICDs, those of skill in the art (e.g., including cardiologists, electrophysiologists, medical doctors, biomedical engineers, medical device specialists—including designers and developers therefor, and the like) appreciate that a vast amount of prior art has been generated as science and medicine continue to advance and converge toward more safe and efficacious methods of providing therapy to terminate potentially lethal episodes of cardiac arrhythmia. The inventor hereof contributes to the advancement and convergence of defibrillation by describing, depicting and claiming the following embodiments and aspects of the subject matter he invented.

### BRIEF SUMMARY OF THE INVENTION

The invention provides effective ventricular defibrillation, defibrillation methods and systems that, according to certain embodiments of the invention, do not require direct contact with excitable myocardial tissue. For example, such embodiments only optionally include a defibrillation electrode disposed within, on or about a heart (e.g., devoid of endocardial, epicardial or pericardial electrodes for example disposed in contact with a portion of the SVC, right ventricle or the like. These embodiments are thus relevant to subjects contra-indicated for medical electrical leads disposed in the right atrium (RA) and right ventricle (RV), as is the case for subjects having an artificial right sided atrio-ventricular valve (i.e., tricuspid valve). Also subjects who already have an implanted and operative RA-RV pacing lead can benefit from the teaching of the invention. That is, the existing pacing lead does not need to be removed and may remain functional without requiring placement of a second lead in the RA-RV.

In a prophylactic application, for example to protect a subject from sudden cardiac death, an ICD system according to the invention can be placed completely in the subcutaneous or submuscular position without the need to place leads or electrodes in the cardiovascular system.

Such a system, using a dorsal subcutaneous array electrode instead of a SVC electrode, has been described in computer simulations with the same model as described above. The invention results in a configuration with the least invasive (in vascular terms) approach, which may be beneficial to the patient. The invention may also be applied in situations where the RV defibrillation electrode has become ineffective, by component failure or other cause. In case the RV defibrillation lead is also provided with a SVC defibrillation electrode, the latter may be used in combination with precordial electrode according to this invention. The invention could also be applied when the ICD device cannot be placed in the left subclavicular area. Right sided or abdominal implantation of the Active Can ICD device may result in ineffective defibrillation capacity of the system. Here the use of precordial electrodes and an inactive Can could help.

The inventions solves the problem if, for example during open chest surgery, epicardial defibrillation electrode placement is not feasible because of large size of the electrodes presence of coronary bypasses or other factors that limit or prohibit or render impractical to use either epicardial or endocardial leads. During this procedure smaller pacing and sensing electrodes can be placed and used for pacing and sensing in the defibrillation system.

One set of embodiments of the invention provides a variety of configurations for delivering cardioversion/defibrillation therapy with a vector of energy controlled by operative circuitry of a non-active can, or canister housing, type ICD. The ICD can be conveniently disposed in a surgically created pocket formed in one of a variety of locations of a patient's abdomen, pectoral region, or the like. The ICD electrically couples to one or more high voltage electrodes (e.g., coil type, patch type, array type, etc.) and at least one electrode disposed within a portion of the SVC part of the vasculature of a patient thus providing a number of clinician-selectable defibrillation vectors between the electrodes tailored for the shape, or contour, of a given patient's heart. The one or more high voltage electrode can include a set of coil electrodes disposed in a precordial orientation relative to a patient's heart that provides several different therapy delivery vectors between the SVC electrode and one or more of the precordial coil electrodes.

In another embodiment of the invention, a high voltage (e.g., coil-type) electrode is disposed within a portion of the SVC part of the vasculature of a patient and in electrical communication with an active ICD can disposed in a precordial position. The SVC coil electrode can be disposed in a relatively “high” location relative to the RA of a patient in order to enhance the therapy delivery vector between the SVC coil and the active can of a precordial ICD.

In yet another embodiment of the invention an active canister portion of a precordial ICD couples a therapy delivery vector between or among the active can, an RV coil electrode, and/or an appropriately located SVC electrode (e.g., disposed in a relatively ‘low’ orientation). As in the former and other embodiments, the precordial ICD can be implanted in a subcutaneous or submuscular location in order to enhance the electrically communication between and/or among the subcutaneous or submuscular precordial-oriented ICD canister, the RV coil electrode and/or the SVC electrode coil.

Another embodiment of the present invention includes an active can, or canister housing, of a precordial ICD providing therapy delivery vectors between the precordial ICD, an SVC coil and/or a high voltage electrode coil disposed within a portion of the coronary sinus/coronary vein (CS/CV) of a patient.

In yet another embodiment of the invention, therapy delivery vectors are selected from a precordial active can, or canister housing, of an ICD and a precordial- or subcutaneous-submuscular electrode array of electrodes. The active can ICD electrically couples to at least one discrete coil electrode that forms a part of a dorsal (back) subcutaneous electrode array disposed around a part of the thorax and ribs of a patient.

Another aspect of the invention involves pacing and/or sensing using a precordial electrode, including one or more electrodes disposed inward of a subject's intercoastal thoracic muscles. In related embodiments all the components of a cardiac sensing, pacing and defibrillation system are spaced from the subject's excitable myocardial tissue.

Inherent in such methods, components and systems the invention is typically embodied in a processor-based implantable medical device operated pursuant to instructions stored on computer readable media. The invention thus includes all such media now known and later developed.

The foregoing embodiments of the invention merely introduce the subject matter of the present invention and although each is depicted and described herein other aspects and embodiments of the invention are fully intended to fall within the breadth and scope of the present invention and are expressly covered hereby.

### BRIEF DESCRIPTION OF THE DRAWINGS

FIG. 1 depicts in schematic format a prior art (i.e., “standard arrangement”) for an implantable cardioverter-defibrillator (ICD) having an optional high voltage (e.g., coil-type) electrode disposed within a portion of the superior vena cava (SVC) part of the vasculature of a patient.

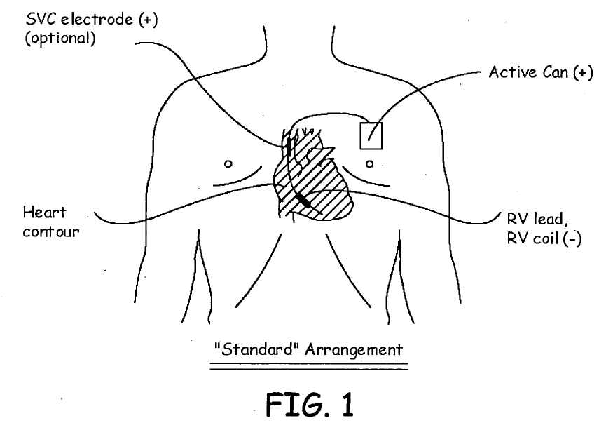

FIG. 2 depicts in schematic format an embodiment of the invention (i.e., “INVENTION”) for a variety of configurations for a non-active can, or canister housing, for an ICD having one or more high voltage (e.g., coil-type) electrodes and at least one electrode disposed within a portion of the SVC part of the vasculature of a patient thus providing a number of clinician-selectable defibrillation vectors given the shape, or contour, of a given subject's heart.

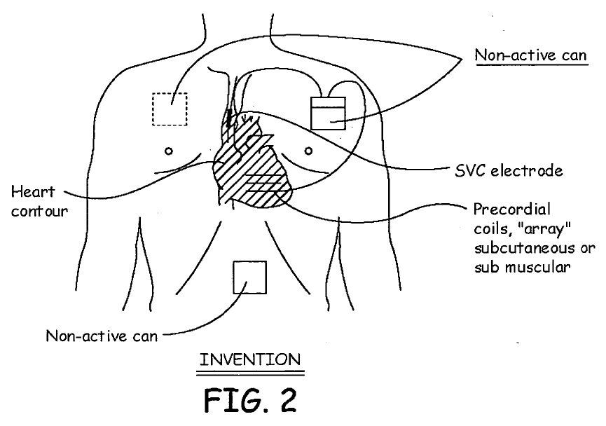

FIG. 3 depicts in schematic format an embodiment of the invention (i.e., “INVENTION”) including a high voltage (e.g., coil-type) electrode disposed within a portion of the SVC part of the vasculature of a subject and in electrical communication with one of an active can, or canister housing, for an ICD disposed in a precordial position of a subject.

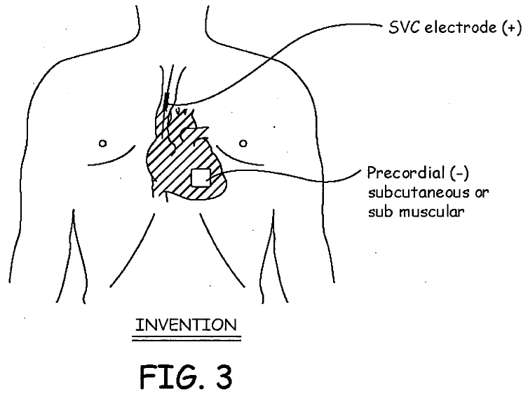

FIG. 4 depicts an alternative application of invention wherein an SVC electrode (e.g., disposed in a relatively ‘low’ orientation) electrically couples to one of an active can, or canister housing, for an ICD and a subcutaneous or submuscular precordial-oriented electrode or array.

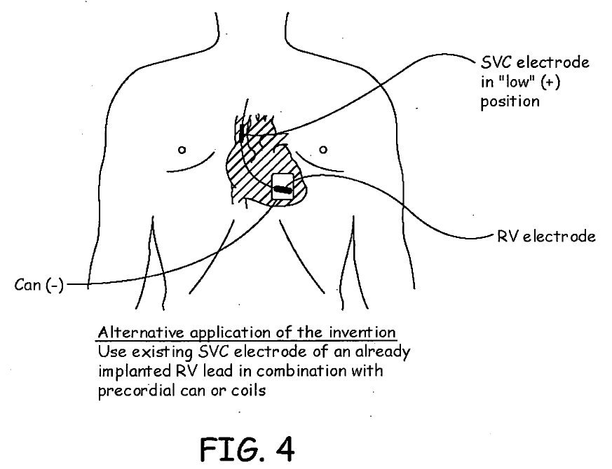

FIG. 5 depicts an alternative application of the invention wherein an active can, or canister housing, for an ICD provides defibrillation vectors in communication with an SVC electrode or a coronary sinus/coronary vein-oriented electrode.

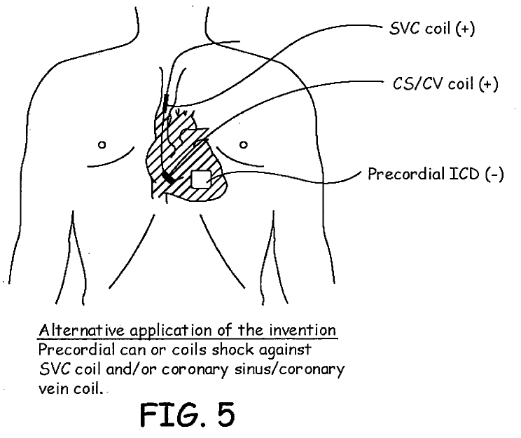

FIG. 6 depicts one of a precordial active can, or canister housing, of an ICD and a precordial- or subcutaneous-submuscular electrode array of electrodes, wherein at least one lateral thoracic (e.g., dorsal or back) electrode is disposed around a part of the thorax of a subject.

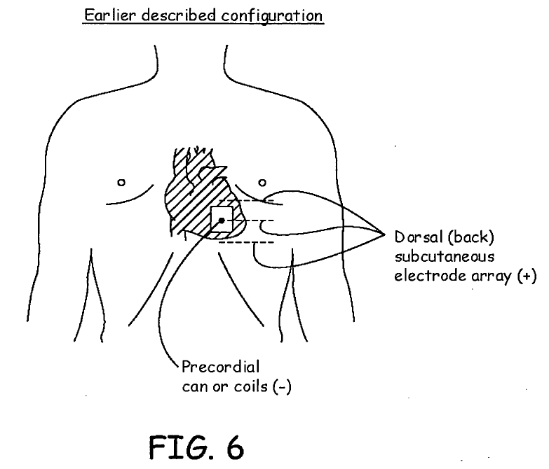

FIG. 7 depicts a table (and sets forth text) captioned, “DFT COMPUTER SIMULATION OF A PRECORDIAL CAN-SUPERIOR VENA CAVA ELECTRODE” that summarizes some relevant data and information.

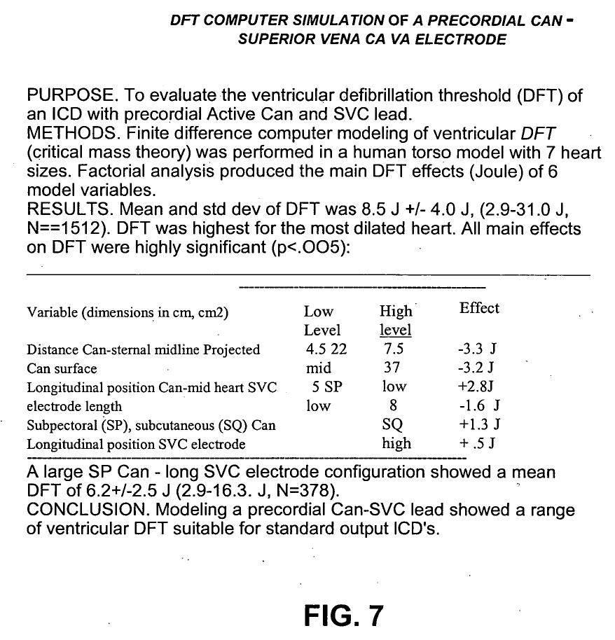

FIG. 8 depicts a table (and sets forth text) captioned, “DFT COMPUTER SIMULATION OF PRECORDIAL COILS—SUPERIOR VENA CAVA ELECTRODES” that summarizes some relevant data and information.

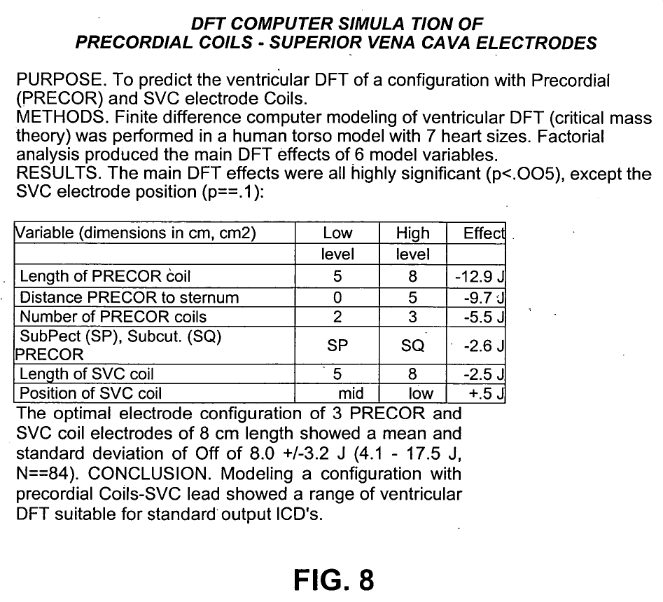

### DETAILED DESCRIPTION OF THE INVENTION

The invention relates to an electrode configuration for an implantable cardioverter defibrillator, not requiring, however allowing, a defibrillation electrode placed in the right ventricle of the heart.

The electrode configuration includes at least one of the following electrodes: a precordial electrode, implanted in the subcutaneous or submuscular tissue in an area in front of the heart on the left side of the chest. Herein the term precordial is intended to have its customary meaning consistent with a definition that includes, without limitation, any position within the margins of the shadow of the left ventricle as can be observed on an antero-posterior chest X-ray. Without limitation, some examples of non-precordial electrode positions include locations within the SVC, any of the coronary sinus and/or coronary venous structures, as well as within a chamber such as the RA or RV. However, according to certain aspects of the invention an SVC electrode (alone as a part of dual defibrillation medical electrical lead) or an electrode disposed partially in the SVC and the RA appears to offer acceptable defibrillation/cardioversion therapy delivery vectors.

The precordial electrode may comprise, without limitation: an active can (conductive portion of a metallic housing of a medical device or ICD); a patch-type electrode (e.g., similar to the types known as epicardial patch electrode); a series of coil- or wire-type electrodes (also known in the art as a “subcutaneous array” or equivalent), characterized by an elongated body with a diameter of preferably less than about three (3) millimeters, which can be tunneled in the subcutaneous or submuscular tissue of a subject; and any combination of the foregoing alternatives.

A reasonable overall set of dimensions for precordial electrodes or combination of electrodes includes approximately six (6) centimeters in the longitudinal (caudal-cranial) direction and about eight (8) centimeters in the lateral direction. Dimensions may of course be varied according to the size of the heart and the thorax, among other factors. Larger electrodes can be utilized and can be expected to result in lower defibrillation shock impedance, which generally leads to lower shock energy, required to defibrillate the heart. The inventor suggests that electrode dimensions exceeding the margins of the heart contour may result in higher defibrillation energy requirement, although still performing according to the teaching of the present invention.

The SVC or RA electrode is preferably placed in a position where the middle part of the electrode is placed at the longitudinal thoracic level of the intersection of the plane formed by the longitudinal axes of the left ventricle and the right ventricle and the elongated center line of the SVC. An effective length of the SVC electrode can be sized with relation to the short axis dimension of the LV, for example, which is typically on the order of about seven (7) to about (10) centimeters.

Pacing and sensing aspects of the invention: Although the design of pacing and sensing electrodes for a precordial defibrillation system is not the primary objective of the invention, pacing and sensing with electrodes specially designed for the precordial system should be considered as part of the invention. Sensing can be done with sensing electrodes incorporated in the precordial active can. Preferably three (3) or four (4) sensing electrodes, with different vectors, should be available in order to be able to select the best combination for detection of ventricular and atrial signals. The selection should be done automatically, because the position and orientation of the can relative to the heart is determined at the time of implant or may change during the period of implantation. In case of precordial coil implantation, the defibrillation coils can be provided with tip or ring electrodes that have sensing performance similar to those of the precordial can.

Stimulation of the heart can be done by the same as the sensing electrodes, which will require more energy than normal pacing electrodes that are implanted inside or directly on the heart. Special electrodes that are pierced through the intercostals muscles and places just outside the pericardium can be used for sensing and pacing at lower energy.

Pacing and sensing can also be performed with any other lead that is permanently implanted. Epicardial electrodes are suitable when placed during an open chest procedure. Endocardial electrodes can also be used. These electrodes may previously have been implanted as part of a pacing or defibrillation system or as part of the configuration described by the invention.

In addition, the electrode configuration may be extended with the use of dorsal electrodes, (in an “array” configuration), preferably with the same polarity as the SVC electrode.

The system using precordial electrodes should have an ICD device with an electrically inactive can or metal housing that could be placed in the subclavicular area or in the abdominal area.

The embodiments of the invention thus described and/or depicted should not be viewed as limiting but rather illustrative of the invention. As those of skill in the art will readily appreciate, variations in components, procedures and steps could be envisaged without departing from the true scope of the invention, which scope is literally defined by the appended claims. The inventor hereby literally claims ownership of all the following structures, methods, systems and computer readable media, including equivalents and insubstantial variations thereof.

1. A method of configuring electrodes for treating tachycardia, comprising:
placing at least a first electrode in a non-precordial position, the first electrode having electrical conductivity with the heart; and
placing a second electrode in a precordial position, the second electrode having electrical conductivity with the heart.

2. A method according to claim 1, wherein the first electrode comprises a metallic coil type electrode.

3. A method according to claim 1, wherein the second electrode comprises at least one of: a metallic coil type electrode, a patch electrode, a subcutaneous electrode array.

4. A method according to claim 1, wherein the non-precordial position comprises one of a location within a portion of a superior vena cava of a subject and a location within a right ventricular chamber of the subject.

5. A method according to claim 1, wherein the precordial position comprises one of a subcutaneous location and a submuscular location.

6. A method according to claim 1, wherein the second electrode comprises at least two metallic coil type electrodes.

7. A method according to claim 6, wherein at least two metallic coil type electrodes comprise a subcutaneous electrode array.

8. A method according to claim 6, wherein the at least two metallic coil type electrodes are located in one of a subcutaneous location and a submuscular location.

9. A method according to claim 1, wherein the first electrode comprises one of an electrically conductive portion of a housing for an implantable medical device and at least two elongated electrodes, and wherein the first electrode is disposed in one of a subcutaneous and a submuscular position of the subject.

10. A method according to claim 9, wherein the implantable medical device comprises an implantable cardioverter-defibrillator.

11. A method according to claim 1, wherein the non-precordial position comprises one of a location within a portion of a coronary sinus of the subject and a location within a portion of a coronary vein of the subject.

12. A system for defibrillating a volume of myocardial tissue from at least a pair of electrodes spaced from the volume of myocardial tissue, comprising:
a first electrode disposed in a precordial location relative to a volume of myocardial tissue; and
a second electrode disposed in a non-precordial location relative to the volume of myocardial tissue.

13. A system according to claim 12, wherein one of the first electrode and the second electrode is disposed in one of a subcutaneous position of a subject and a submuscular position of the subject.

14. A system according to claim 12, wherein one of the first electrode and the second electrode comprises a metallic coil type electrode.

15. A system according to claim 12, wherein one of the first electrode and the second electrode comprises an electrically conducting portion of a housing for an implantable medical device.

16. A system according to claim 12, wherein one of the first electrode and the second electrode comprises a subcutaneous electrode array.

17. A system according to claim 16, wherein the subcutaneous electrode array comprises at least two elongated medical electrical leads and a distal portion of each of the electrical leads comprises at least one electrically conductive coil.

18. A system according to claim 16, wherein the second electrode comprises the subcutaneous electrode array and said second electrode is disposed in a portion of at least one of an anterior lateral thoracic and a posterior lateral thoracic location of a subject.

19. A system according to claim 12, wherein the volume of myocardial tissue comprises a portion of ventricular tissue.

20. A system according to claim 12, wherein both the first electrode and the second electrode comprise at least one elongated coil type electrode.

21. A system according to claim 12, wherein the first electrode comprises at least two metallic coil type electrodes and the second electrode comprises at least two metallic coil type electrodes.

22. A system according to claim 20, further comprising an implantable cardioverter-defibrillator operatively coupled to both the first and the second electrodes.

23. A system according to claim 12, wherein both the first electrode and the second electrode comprise at least one metallic coil type electrode.

24. A system according to claim 12, wherein both the first electrode and the second electrode comprise a patch type electrode.

25. A method of deploying an implantable cardiac defibrillation system, comprising:
deploying a first electrode into a non-precordial location relative to a heart of a subject; and
inserting a second electrode in one of a submuscular and a subcutaneous position and in a precordial location relative to the heart of the subject.

26. A method according to claim 25, wherein the first electrode comprises a metallic coil type electrode.

27. A method according to claim 25, wherein the second electrode comprises at least one of:
a metallic coil type electrode, a patch electrode, a subcutaneous electrode array.

28. A method according to claim 25, wherein the non-precordial position comprises one of a location within a portion of a superior vena cava of a subject and a location within a right ventricular chamber of the subject.

29. A method according to claim 25, wherein the precordial position comprises one of a subcutaneous location and a submuscular location.

30. A method according to claim 25, wherein the second electrode comprises at least two metallic coil type electrodes.

31. A method according to claim 30, wherein at least two metallic coil type electrodes comprise a subcutaneous electrode array.

32. A method according to claim 30, wherein the at least two metallic coil type electrodes are located in one of a subcutaneous location and a submuscular location.

33. A method according to claim 25, wherein the first electrode comprises one of an electrically conductive portion of a housing for an implantable medical device and at least two elongated electrodes, and wherein the first electrode is disposed in one of a subcutaneous and a submuscular position of the subject.

34. A method according to claim 25, wherein the implantable cardiac defibrillation system comprises an implantable cardioverter-defibrillator.

35. A method according to claim 25, wherein the non-precordial position comprises one of a location within a portion of a coronary sinus of the subject and a location within a portion of a coronary vein of the subject.

36. A method according to claim 25, further comprising:
coupling a proximal portion of the first electrode to operative circuitry of an implantable medical device.

37. A method according to claim 36, wherein the implantable cardiac defibrillation system comprises an implantable cardioverter-defibrillator.

38. A method according to claim 37, wherein a conductive portion of the implantable cardioverter-defibrillator comprises the second electrode.

39. A method of delivering cardiac pacing therapy to a heart of a subject, comprising:
sensing cardiac events with at least a pair of electrodes located in one of a submuscular and a subcutaneous position, wherein the at least a pair of electrodes are spaced from excitable heart tissue of a subject; and
delivering a series of timed cardiac pacing stimulus pulses via the at least a pair of electrodes at a sufficient energy level to capture at least one ventricular chamber of the subject.

40. A method according to claim 39, wherein the at least a pair of electrodes are disposed in a precordial position relative to the excitable heart tissue of the subject.

41. A method according to claim 40, wherein the at least a pair of electrodes couple to a surface portion of an implantable medical device.

42. A method according to claim 41, wherein the at least a pair of electrodes couple in a spaced apart relation upon the surface portion of an implantable medical device.

43. A method according to claim 42, wherein the at least a pair of electrodes comprise at least three electrodes.

44. A method according to claim 39, wherein the at least a pair of electrodes are disposed in a location inward of the intercoastal muscles of the thorax of the subject.

45. A method according to claim 40, further comprising at least one defibrillation electrode coupled to the at least a pair of electrodes.

46. A method according to claim 45, wherein the at least a pair of electrodes comprises at least one ring-type electrode.

47. A method according to claim 45, wherein the at least one defibrillation electrode comprises at least one of: a patch type electrode, an elongated coil type electrode, a subcutaneous electrode array.

48. A method according to claim 39, wherein the at least a pair of electrodes comprises at least three electrodes and further comprising:
automatically activating different pairs of the at least three electrodes to render a plurality of sensing vector data sets representative of cardiac activity of the subject; and
comparing at least two of the plurality of sensing vector data sets.

49. A method according to claim 48, further comprising:
utilizing a pair of the different pairs for subsequent sensing of cardiac activity of the subject based at least in part on the comparison of the sensing vector data sets.

50. A method according to claim 48, further comprising:
storing in a memory structure at least one metric related to at least one pair of the different pairs.

51. A method according to claim 48, further comprising:
wirelessly transmitting at least one of the data sets and the results of the comparing step to an external device.

52. A method according to claim 39, further comprising:
storing in a memory structure at least a portion of the sensed cardiac events.

53. A method according to claim 52, wherein the portion of the sensed cardiac events comprises at least one of: a temporal portion of events, a heart rate metric, an arrhythmia metric, an upper rate metric, a lower rate metric, a percent-paced metric, a pacing energy metric, a percent-pacing-capture metric.

54. A method according to claim 39, further comprising:
delivering one of a cardioversion therapy and a defibrillation therapy via the at least one defibrillation electrode in the event that the sensed cardiac events exceed a potentially lethal tachycardia episode threshold.

55. A computer readable medium for storing instructions for performing a method of sensing cardiac activity, delivering cardiac pacing or defibrillation therapy, said medium comprising:
instructions for sensing cardiac events with at least a pair of electrodes located in one of a submuscular and a subcutaneous position and spaced from excitable heart tissue of a subject; and
instructions for delivering a series of timed cardiac pacing stimulus pulses via the at least a pair of electrodes at a sufficient energy level to capture at least one ventricular chamber of the subject.

56. A medium according to claim 55, wherein the at least a pair of electrodes are disposed in a precordial position relative to the excitable heart tissue of the subject.

57. A medium according to claim 56, wherein the at least a pair of electrodes couple to a surface portion of an implantable medical device.

58. A medium according to claim 57, wherein the at least a pair of electrodes are coupled in a spaced apart relation upon the surface portion of an implantable medical device.

59. A medium according to claim 58, wherein the at least a pair of electrodes comprise at least three electrodes.

60. A medium according to claim 55, wherein the at least a pair of electrodes are disposed in a location inward of the intercoastal muscles of the thorax of the subject.

61. A medium according to claim 56, further comprising at least one defibrillation electrode coupled to the at least a pair of electrodes.

62. A medium according to claim 61, wherein the at least a pair of electrodes comprises at least one ring-type electrode.

63. A medium according to claim 61, wherein the at least one defibrillation electrode comprises at least one of: a patch type electrode, an elongated coil type electrode, a subcutaneous electrode array.

64. A medium according to claim 55, wherein the at least a pair of electrodes comprises at least three electrodes and further comprising:
instructions for automatically activating different pairs of the at least three electrodes to render a plurality of sensing vector data sets representative of cardiac activity of the subject; and
instructions for comparing at least two of the plurality of sensing vector data sets.

65. A medium according to claim 64, further comprising:
instructions for electrically connecting a pair of the different pairs for subsequent sensing of cardiac activity of the subject based at least in part on the comparison of the sensing vector data sets.

66. A medium according to claim 64, further comprising:
instructions for storing in a memory structure at least one metric related to at least one pair of the different pairs.

67. A medium according to claim 64, further comprising:
instructions for wirelessly transmitting at least one of the data sets and the results of the comparing step to an external device.

68. A medium according to claim 55, further comprising:
instructions for storing in a memory structure at least a portion of the sensed cardiac events.

69. A medium according to claim 55, further comprising:
means for delivering one of a cardioversion therapy and a defibrillation therapy via the at least one defibrillation electrode in the event that the sensed cardiac events exceed a potentially lethal tachycardia episode threshold.

70. A system for defibrillating a volume of excitable myocardial tissue, comprising:
means for sensing cardiac events with at least a pair of electrodes located in one of a submuscular and a subcutaneous position and spaced from excitable heart tissue of a subject; and
means for delivering a series of timed cardiac pacing stimulus pulses via the at least a pair of electrodes at a sufficient energy level to capture at least one ventricular chamber of the subject.

71. A system according to claim 70, wherein the at least a pair of electrodes are disposed in a precordial position relative to the excitable heart tissue of the subject.

72. A medium according to claim 71, wherein the at least a pair of electrodes couple to a surface portion of an implantable medical device.

73. A medium according to claim 72, wherein the at least a pair of electrodes are coupled in a spaced apart relation upon the surface portion of an implantable medical device.

74. A medium according to claim 73, wherein the at least a pair of electrodes comprise at least three electrodes.

75. A system according to claim 70, wherein the at least a pair of electrodes are disposed in a location inward of the intercoastal muscles of the thorax of the subject.

76. A medium according to claim 71, further comprising at least one defibrillation electrode coupled to the at least a pair of electrodes.

77. A medium according to claim 76, wherein the at least a pair of electrodes comprises at least one ring-type electrode.

78. A medium according to claim 76, wherein the at least one defibrillation electrode comprises at least one of: a patch type electrode, an elongated coil type electrode, a subcutaneous electrode array.

79. A system according to claim 55, wherein the at least a pair of electrodes comprises at least three electrodes and further comprising:
means for automatically activating different pairs of the at least three electrodes to render a plurality of sensing vector data sets representative of cardiac activity of the subject; and
means for comparing at least two of the plurality of sensing vector data sets.

80. A medium according to claim 79, further comprising:
means for electrically connecting a pair of the different pairs for subsequent sensing of cardiac activity of the subject based at least in part on the comparison of the sensing vector data sets.

81. A medium according to claim 79, further comprising:
means for storing in a memory structure at least one metric related to at least one pair of the different pairs.

82. A medium according to claim 79, further comprising:
means for wirelessly transmitting at least one of the data sets and the results of the comparing step to an external device.

83. A system according to claim 70, further comprising:
means for storing in a memory structure at least a portion of the sensed cardiac events.

84. A system according to claim 70, further comprising:
means for delivering one of a cardioversion therapy and a defibrillation therapy via the at least one defibrillation electrode in the event that the sensed cardiac events exceed a potentially lethal tachycardia episode threshold.
